(19) United States
(12) Patent Application Publication
(10) Pub. No.: US 2002/0030164 A1
(43) Pub. Date: Mar. 14, 2002

(54) Charged particle beam irradiation equipment

(75) Inventors: Hiroshi Akiyama, Hitachiota (JP); Koji Matsuda, Hitachi (JP); Kazuo Hiramoto, Hitachiota (JP); Kohei Kato, Hitachi (JP); Kazumichi Suzuki, Mito (JP)

Correspondence Address: Crowell & Moring LLP, Evenson, Mckeown, Edwards & Lenehan, Intellectual Property Law Group, 1200 G St., N.W., Suite 700, Washington, DC, 20005, US

(73) Assignee: Hitachi, Ltd.

(21) Appl. No.: 09/949,849

(22) Filed: Sep. 12, 2001

(30) Foreign Application Priority Data
Sep. 12, 2000 (JP) 2000-314419

(51) Int. Cl. A61N 5/00; G21G 5/00

(52) U.S. Cl. 250/492.1

(57) ABSTRACT

A charged particle beam irradiation equipment enable to reduce the cost in case of irradiating the beam to the target object in plural directions.

The irradiation nozzle parts 105a to 105c comprising a wobbler electromagnet, a scatterer, a range modulator, a patient collimator and a patient bolus are shared by plural irradiation ports 102a and 102b providing charged particle beams to the irradiation object in plural different directions.

### BACKGROUND OF THE INVENTION

The present invention relates to a charged particle beam irradiation equipment for irradiating a charged particle beam to the diseased part of the patient for therapy.

Cancer therapy by irradiating a charged particle beam (hereinafter referred to as beam) accelerated up to high energy gets a lot of attention in recent years. As for the irradiation equipment for irradiating the beam, an irradiation equipment having a structure shown in FIG. 7 is well known. The irradiation equipment shown in FIG. 7 can irradiate the beam to the patient in the direction vertical or horizontal to the floor 704. This way of altering the direction for irradiating the beam is aimed for minimizing the damage to the normal tissue (regions other than the diseased part) of the patient.

In case of irradiating the beam in the direction vertical to the floor 704, the deflection electromagnets 701a to 701c and the quadrupole electromagnets 702a to 702d are made excited at first, and then, while this excitation continues, the beam accelerated by the accelerator (not shown) such as synchrotron is led to the deflection electromagnet 701a. The beam introduced into the deflection electromagnet 701a is deflected by the deflection electromagnets 701a to 701c, and its tune is adjusted by the quadrupole electromagnets 702a to 702d, and then the beam is introduced to the irradiation nozzle 703a. The irradiation nozzle 703a is formed by the wobbler electromagnet or the patient bolus, and after the beam diameter and energy of the beam introduced into the irradiation nozzle 703a are adjusted, then the beam is irradiated to the diseased part of the patient.

In case of irradiating the beam in the direction horizontal to the floor 704, the beam is introduced from the accelerator while the deflection electromagnet 701a is not made excited and the quadrupole electromagnets 702e and 702f are made excited. The introduced beam is not deflected due to the non-excitation state of the deflection electromagnet 701a, but goes straight, and then, after its tune is adjusted by the quadrupole electromagnets 702e and 702f, the beam is introduced into the irradiation nozzle 703b. The irradiation nozzle 703b is formed by the wobbler electromagnet or the patient bolus similar to the irradiation nozzle 703a, and after the beam diameter and energy of the beam introduced into the irradiation nozzle 703b are adjusted, then the beam is irradiated to the diseased part of the patient.

As described above, the conventional irradiation equipment irradiates the beam in plural directions to the diseased part of the patient.

As described above, the conventional irradiation equipment has plural irradiation nozzles dedicated to irradiate the individual beams in the different directions. Thus, for example, in case of irradiating the beams in four different directions, four independent irradiation nozzles are required. It is preferable in the beam irradiation therapy to make the number of irradiation directions as large as possible in order to minimize the damage to the normal tissue. As described above, the larger the number of directions in which the beam is irradiated in the conventional irradiation equipment is, the larger the number of irradiation nozzles is, which leads to an increase in the cost for the irradiation equipment.

### SUMMARY OF THE INVENTION

The present invention provides a charged particle beam irradiation equipment enabling to reduce the cost in case of irradiating the beam in plural directions.

The present invention for attaining the above object is characterized as a charged particle beam irradiation equipment having plural irradiation ports for providing a charged particle beam in plural different directions to an irradiation target to be irradiated with the charged particle beam, and an equipment for adjusting the characteristic of the charged particle beam provided from the irradiation port in responsive to the irradiation target, in which the equipment for adjusting the characteristic of the charged particle beam is shared by plural irradiation ports. In this context, the irradiation target is a patient having medical treatment, and the component for adjusting the characteristic of the charged particle beam includes substantially a wobbler electromagnet, a scatterer, a range modulator, a patient collimator and a patient bolus.

In case of irradiating the charged particle beam in plural directions to the irradiation target by using plural irradiation ports, as the equipment for adjusting the characteristic of the charged particle beam is shared by plural irradiation ports, it will be appreciated that the number of components for the equipment used for adjusting the characteristic of the charged particle beam can be reduced in comparison with such a case that the those components are installed for the individual irradiation ports, and thus, the cost of the irradiation equipment can be downsized.

### BRIEF DESCRIPTION OF THE DRAWINGS

FIG. 1 is a structure of the charged particle beam irradiation equipment in one preferred embodiment of the present invention.

FIG. 2 is a structure of the therapy system using the charged particle beam irradiation equipment shown in FIG. 1.

FIG. 3 is a structure of the therapy planning equipment for building the therapy plan.

FIG. 4 is a structure of the irradiation control equipment for controlling the charged particle beam irradiation equipment shown in FIG. 1.

FIG. 5 is a structure of the irradiation nozzle parts 105a to 105c shown in FIG. 1.

FIG. 6 is a structure of the irradiation nozzle part of the charged particle beam irradiation equipment in another embodiment of the present invention.

FIG. 7 is a structure of the conventional charged particle beam irradiation equipment.

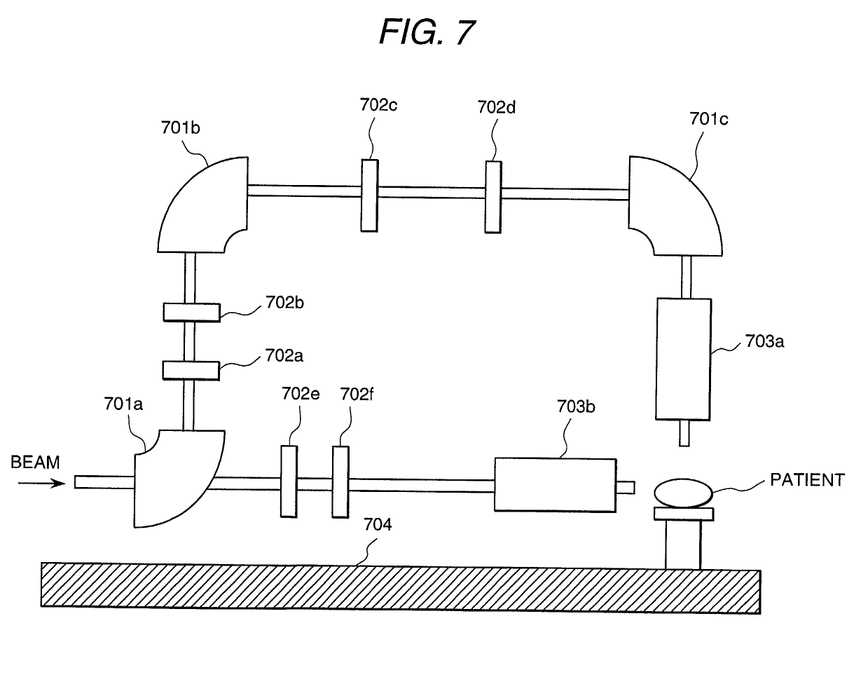

### DESCRIPTION OF THE PREFERRED EMBODIMENTS OF THE INVENTION

Now, referring to the attached figures, the preferred embodiments of the present invention will be described below.

(Embodiment 1)

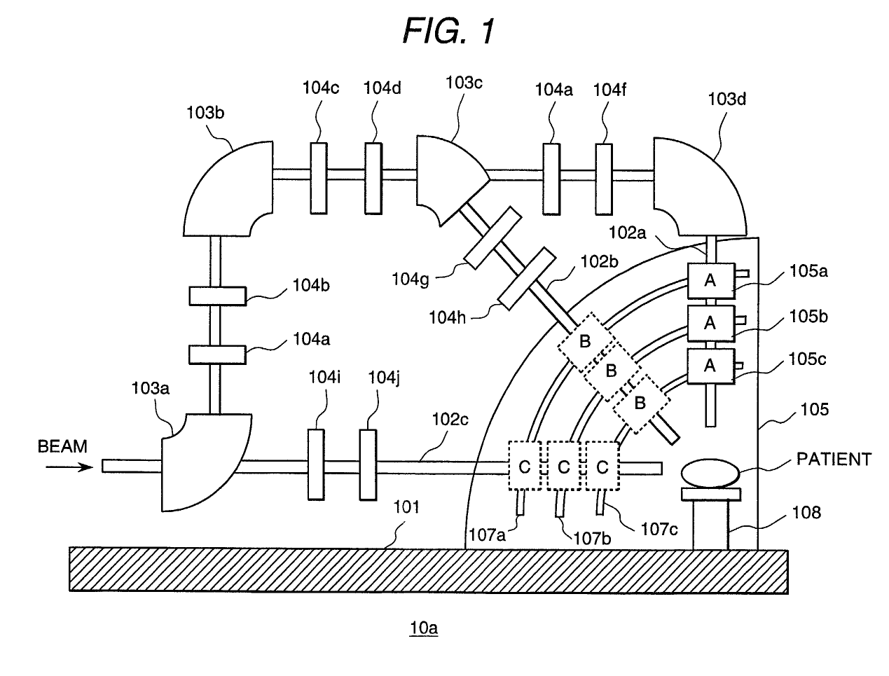

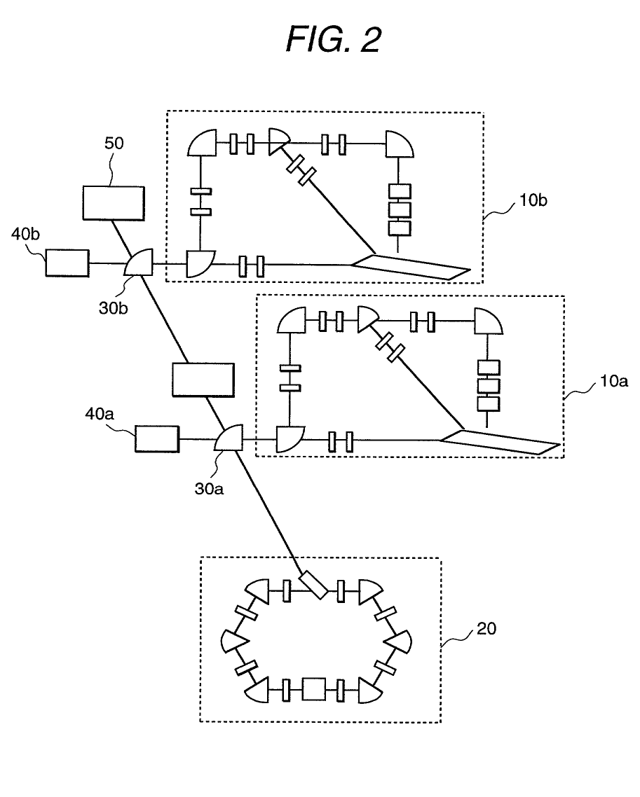

FIG. 1 illustrates a structure of the charged particle beam irradiation equipment in the preferred embodiment of the present invention, and FIG. 2 illustrates a structure of the therapeutic system having the charged particle beam irradiation equipment shown in FIG. 1.

At first, the therapeutic system shown in FIG. 2 is described. In the synchrotron 20 shown in FIG. 2, the charged particle beam (hereinafter referred to as beam) is accelerated and then emitted, and the emitted beam is led to the switching electromagnet 30b. Though a synchrotron is used as the accelerator in this embodiment, it is allowed to use another kind of accelerator such as cyclotron and linear accelerator. When the switching electromagnet 30a is excited, it deflects and introduce the beam to the irradiation equipment 10a, and when it is not excited, it makes the beam go straight without deflection. The switching electromagnet 30a is excited by the electric power supplied by the power supply 40a, and the electric power supplied from the power supply 40a is controlled by the switch on the control panel operated by the operator. When operating the irradiation equipment 10a for the therapy for the patient, the electric power is supplied from the power supply 40a to the switching electromagnet 30a though the switch operation of the operator; and when the therapy for the patient is not provided in the irradiation equipment 10a, the electric power is not supplied from the power supply 40a to the switching electromagnet 30a by the switch operation of the operator. The therapy for the patient is provided by irradiating the guided beam to the diseased part of the patient in the irradiation equipment 10a, its detail will be described after.

When providing a therapy in the irradiation equipment 10b, the electric power supply to the switching electromagnet 30a is made suspended and the electric power is supplied to the switching electromagnet 30b. As in the case for supplying the electric power to the switching electromagnet 30a, the electric power to the switching electromagnet 30b is supplied by controlling the power supply 40b operated by the switch operation by the operator. By suspending the electric power supply to the switching electromagnet 30a and supplying the electric power to the switching electromagnet 30b, the beam goes straight through the switching electromagnet 30a in a non-excited state, and then deflected by the switching electromagnet 30b in a excited state, and introduced into the irradiation equipment 10b. The therapy in the irradiation equipment 10b will be described later. In case that the therapy is not provided at the irradiation equipment 10a and 10b, the ejection of the beam from the synchrotron 20 may be suspended or the beam may be led to the beam dump 50 by making the state of the switching electromagnets 30a and 30b unexcited.

Next, the irradiation of the beam in the irradiation equipment 10a is described in detail. 10a in the embodiment of the present invention shown in FIG. 1 is so configured as to irradiate the beam in three directions to the patient. The vacuum duct 102a disposed to be vertical to the floor 101, the vacuum duct 102b disposed to be inclined by 40° to the floor 101, and the vacuum duct 102c disposed to be horizontal to the floor 101 are installed, and the direction of the beam can be altered by selecting the vacuum duct for guiding the beam. In this embodiment, the vacuum ducts 102a to 102c are designated irradiation ports 102a to 102c.

In case of irradiating the beam in the direction vertical to the floor 101, the deflection electromagnets 103a, 103b and 103d as well as the quadruple electromagnets 104a to 104f are made excited. At the same, the deflection electromagnet 103c is surely made unexcited. As described before the beam ejected from the synchrotron is introduced into the deflection electromagnet 103a of the irradiation equipment 10a, and is deflected by the deflection electromagnets 103a, 103b and 103d, and its tune is adjusted by the quadrupole electromagnets 104a to 104f, and then the beam is introduced to the irradiation nozzle 102a. As the deflection electromagnet 103c is not excited, the beam goes straight without reflected by the deflection electromagnet 103c.

After the beam is put out from the irradiation port 102a, the beam is irradiated though the irradiation nozzle to the patient lying on the therapy bed 108. The irradiation nozzle is separated into three irradiation nozzle parts 105a to 105c and they are enabled to move on the rails 107a to 107c, respectively, and thus, the irradiation nozzle parts 105a to 105c are adjusted to locate at the positions (shown by the letter A in the figure) on the rails 107a to 107c where the beam ejected from the irradiation port 102a passes. Limit switches are preset at the positions where the beam ejected from the irradiation port 102a, and the irradiation nozzle parts 105a to 105c are made move to the limit switches, respectively by controlling the driving equipment (not shown) installed at the irradiation nozzle parts 105a to 105c. Thus, this embodiment uses the rails 107a to 107c, their limit switches and the driving equipment as the mechanism for moving the irradiation nozzle. By irradiating the beam through the irradiation port 102a so formed with the above configuration, the beam can be irradiated to the patient in the direction vertical to the floor 101.

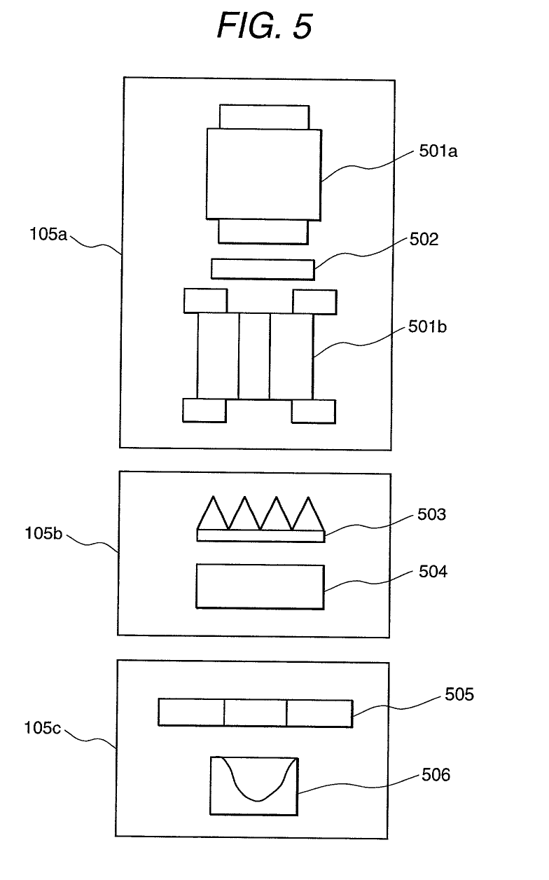

FIG. 5 illustrates the structure of the irradiation nozzle parts 105a to 105c. The irradiation nozzle part 105a is composed of the wobbler electromagnets 501a and 502b and the scatterer 502, and the irradiation nozzle part 105b is composed of the range modulator 503 and the dose monitor 504. The irradiation nozzle part 105c is composed of the patient collimator 505 and the patient bolus 506. As for the function of the individual components in FIG. 5, the wobbler electromagnets 501a and 502b are electromagnets for expanding the beam diameter by scanning the beam in a circle, the scatterer 502 is a component for expanding the beam diameter by scattering the beam. The range modulator 503 is a component for expanding the energy spectrum of the beam by reducing the peak energy of the beam and thus making the width of Bragg peak wider, and the dose monitor 504 is a component or measuring the dose of the beam. The patient collimator 505 is a component for shaping the shape of the beam in the vertical direction (a direction vertical to the progressive direction of the beam) in order to adapt its shape with the shape of the diseased part, and the patient bolus 506 is a component for adjusting the energy of the beam in order to accommodate the shape of the bottom of the diseased part in the depth direction of the diseased part (the progressive direction of the beam).

Next, what is described is a case that the beam is irradiated in the direction inclined by 45° to the floor 101. At first the irradiation nozzle parts 105a to 105c are made move to the positions (shown by the letter B in the figure) where the beam ejected from the irradiation port 102b passes. Then, the deflection electromagnets 103a to 103c and the quadruple electromagnets 104a to 104d, 104g and 104h are made excited. The beam introduced by the deflection electromagnets 103a is further introduced to the irradiation port 102b by the deflection electromagnets 103a to 103c and the quadruple electromagnets 104a to 104d, 104g and 104h, and finally irradiated to the patient through the irradiation port 102b and the irradiation nozzle parts 105a to 105c. By irradiating the beam through the irradiation port 102b so formed with the above configuration, the beam can be irradiated to the patient in the direction inclined by 45° to the floor 101.

Still consider the case in which the beam is irradiated in the direction horizontal to the floor 101. At first, the irradiation nozzle parts 105a to 105c are made move to the positions (shown by the letter C in the figure) where the beam ejected from the irradiation port 102c passes. Then, the quadruple electromagnets 104i and 104j are made excited. At the same, the deflection electromagnets 103a is surely made unexcited. The beam introduced into the deflection electromagnets 103a is not deflected but goes straight through the deflection electromagnets 103a, and then, after its tune is adjusted by the quadrupole electromagnets 104i to 104j, and then the beam is introduced to the irradiation nozzle 102c, and finally, the beam is irradiated to the patient through the irradiation port 102c and the irradiation nozzle parts 105a to 105c. Thus, by irradiating the beam through the irradiation port 102c so formed with the above configuration, the beam can be irradiated to the patient in the direction horizontal to the floor 101.

As described above, the irradiation equipment 10a of the present invention can irradiate the beam in the three directions. In addition to the functionality for enabling the irradiation of the beam in the three direction with three irradiation ports in the irradiation equipment 10a of this embodiment, it will be appreciated that the irradiation beam direction to the patient can be adjusted by make the therapy bed slanted for changing the orientation of the patient. As the structure of the irradiation equipment 10b is the same as the structure of the irradiation equipment 10a, its detail deception is not repeated here.

Next, a definite control method of the irradiation equipment 10a is described below. A number of components forming the irradiation equipment 10a are controlled based on the predefined therapy plan, and a method for building a therapy plan is described at first.

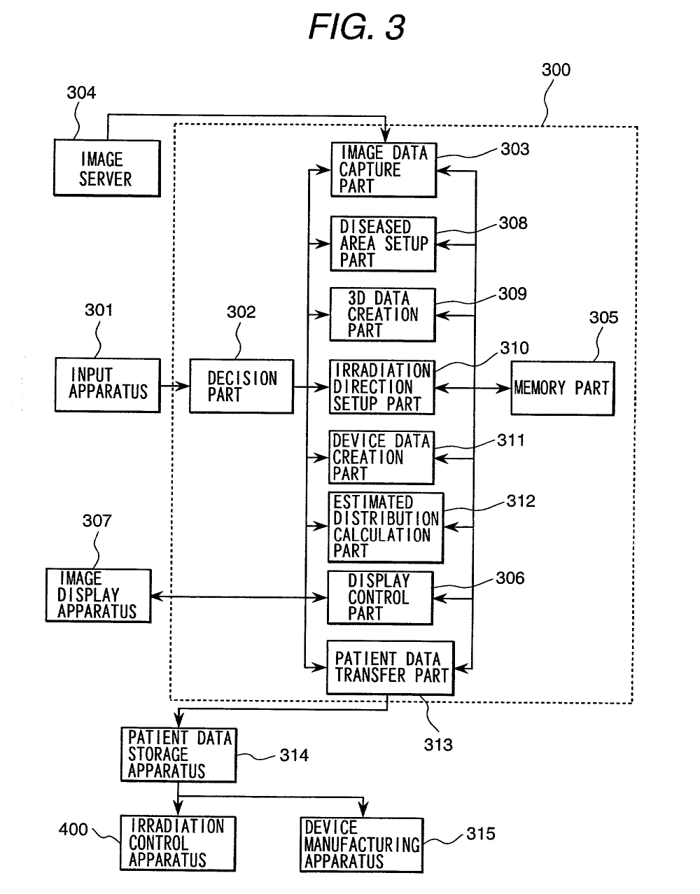

FIG. 3 illustrates a structure of the therapy planning equipment for building a therapy plan. At first, by using the input equipment 301, the operator inputs the information for identifying the patient to be cured (for example, the name of the patient and/or the number precedently assigned to the patient, designated patient identification information) into the therapy planning equipment 300. In the therapy planning equipment 300, the patient identification information given by the input equipment is forwarded to the decision part 302, and the decision part 302 provides the patient identification information to the image data capture part 303. The image capture part 303 captures the image data of the patient specified the given patient identification information from the image server 304. The image data means a tomographic image data obtained by the X-ray CT scanner for radio isotopic imaging of the diseased part of the patient, and the image server 304 stores plural image data, each corresponding to the individual patient identification information, before hand. The image data captured in the image data capture part 303 is stored in the memory part 305 as well as provided to the display control part 306. The display control part 306 presents the tomographic image of the diseased part of the patient to be displayed on the image display equipment 307 in responsive to the provided image data.

The operator inputs the information such as the diseased part, the irradiation target position, the profile line of the patient, the critical organ and the position identification markers on the tomographic image displayed on the image display equipment 307 by using the input equipment 301. The input information is provided through the decision part 302 to the diseased area setup part 308, and then the diseased area setup part 308 stores the input information linked with the image data to the memory part 305. Next, the operator inputs the 3D image display instruction from the input equipment 301, and then the provided 3D image display instruction is sent through the decision part 302 to the 3D data creation part 309. The 3D data creation part 309, responding to the 3D image display 14 instruction, generates 3D body data from the tomographic image data stored in the memory part 305, and stores the generated 3D body data into the memory part 305. The display control part 306 reads out the image data and the 3D body data, both stored in the memory part 305, and then displays the tomographic image and the 3D body image generated based on those data on the image display equipment 307.

The operator inputs the irradiation direction of the beam to the patient by using the input equipment 301 while he or she is viewing the tomographic image and the 3D body image generated displayed on the image display equipment 307. As the beam is irradiated in plural directions to the diseased part in this embodiment, plural irradiation directions are specified with their irradiation orders by the operator. The plural irradiation directions provided in the above manner are forwarded through the decision part 302 to the irradiation direction setup part 310, and then the irradiation direction setup part 310 determines the irradiation port to be used and the inclination of the therapy bed individually for each of the plural irradiation directions. The determined irradiation port and the determined inclination of the therapy bed, both linked to each of plural irradiation directions, are stored into the memory part 305. In case that the irradiation in the irradiation direction specified by the operator can not be available, if any, such a message reporting that the specified direction is not available for irradiation operation is made displayed on the image display equipment 307. By means that the irradiation direction specified by the operator is displayed as an arrow superimposed onto the tomographic image and the 3D body image at the image display equipment 307, and that the tomographic image and the 3D body image at the image display equipment 307 may be displayed as inclined views when the therapy bed is made inclined in order to change the orientation of the patient, the operator can be recognize more easily the irradiation direction.

The device data creation part 311 calculates the depth position of the diseased part, the thickness of the diseased part, the 2D shape of the diseased part and the shape of the bottom part of the diseased part, those viewed in the irradiation direction stored in the memory part 305, by referring to the 3D body data stored in the memory part 305.

The device data such as the energy of the beam ejected from the synchrotron, the current value to be applied to the wobbler electromagnet, the thickness of the scatterer, the shape of the range moculator, the shape of the patient collimator and the shape of the patient bolus are obtained by the calculation results and made stored into the memory part 305. This set of device data is calculated for the individual irradiation direction.

Next, the radiation dose distribution calculation part 312 calculates the radiation dose distribution for the individual irradiation direction from the radiation direction, the device data and the 3D body data, each stored in the memory part 305, and its computational result for the radiation dose is put out to the display control part 306. The display control equipment 306 makes the image display equipment 307 display the radiation dose distribution in the patient body based on the provided computational result for the irradiation dose. If the operator judges that the displayed radiation dose distribution is good, the operator could accept the computational result, but if the operator recognizes any problem such that excess radiation dose is applied to some important organ, the operator could attempt to repeat the calculation of the radiation dose distribution by altering the irradiation direction until a satisfactory radiation dose distribution can be obtained.

The irradiation port, the inclination of the therapy bed and the device data corresponding to the radiation dose distribution accepted by the operator are made linked to the patient identification information for the corresponding irradiation direction, individually, and forwarded from the memory part 305 to the patient data transfer part 313, and then, the patient data transfer part 313 transfers the patient identification information, the irradiation direction, the irradiation port, the inclination of the therapy bed and the device data to the patient data storage equipment 314. A set of data including the patient identification information, the irradiation direction, the irradiation port, the inclination of the therapy bed and the device data is designated patient data. The input patient data is stored in the patient data storage equipment 314, and the patient data is provided in responsive to the request issued by the irradiation control equipment 400 and the device manufacturing equipment 315. The device manufacturing equipment 315 manufactures the scatterer, the range modulator, the patient collimator and the patient bolus based on the thickness of the scatterer, the shape of the range modulator, the shape of the patient collimator and the shape of the patient bolus in the patient data stored in the patient data storage equipment 314. The irradiation control equipment 400 will be described later.

As described above, the therapy plan is established by the therapy planning equipment 300 (which means that the patient data is obtained), and then the therapy for the patient is conducted according to this patient data (therapy plan).

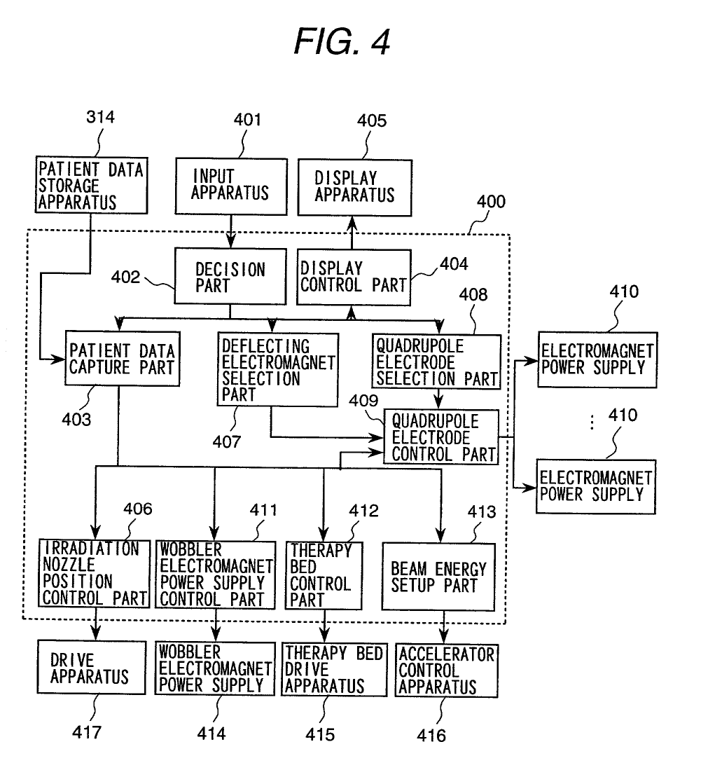

FIG. 4 illustrates the structure of the irradiation control equipment 400. In the irradiation control equipment 400, at first, the operator inputs the patient identification information for the patient to be cured, and selects the irradiation equipment to be used, both by using the input equipment 401, and then the patient identification information and the information specifying the selected irradiation equipment are put into the decision part 402 (in this case, assume that the irradiation equipment 10a is selected). The patient identification information put into the decision part 402 is forwarded to the patient data capture part 403, and then the patient data capture part 403 captures the patient data corresponding to the provided patient identification information from the patient data storage equipment 314. The irradiation direction for the first irradiation operation is selected from the patient data captured by the patient data capture part 403 is put into the display control part 404, and then the display control part 404 makes the display equipment 405 display its irradiation direction. The operator arranges the manufactured parts of the scatterer, the range modulator, the patient collimator and the patient bolus at the individual irradiation nozzles in considering the irradiation directions displayed on the display equipment 405.

The irradiation port (assumed to be the irradiation port 102a, in this embodiment) corresponding to the irradiation direction for the first irradiation operation in the patient data captured by the patient data capture part 403 is supplied to the irradiation nozzle position control part 406, the deflecting electromagnet selection part 407 and the quadrupole electromagnet selection port 408. The irradiation nozzle position control part 406 control the drive equipment 417 installed at the individual irradiation nozzle based on the specified irradiation port in order to locate the irradiation nozzle at the position where the beam ejected from the irradiation port 102a passes through. The information specifying the irradiation equipment is also put into the deflecting electromagnet selection part 407 from the decision part 402, and then the deflecting electromagnet selection part 407 selects the deflecting electromagnets (deflecting electromagnets 103a, b and d, in this case) required to be activated among the deflecting electromagnets forming the irradiation equipment 10a when using the irradiation port 102a, and provides the information specifying those selected deflecting electromagnets to the electromagnet power control part 409. The energy of the beam ejected from the synchrotron in the patient data is also put into the electromagnet power control part 409, and the quadrupole electromagnet selection part 408 selects the quadrupole electromagnets (quadrupole electromagnets 104a to 104f, in this case) forming the irradiation equipment 10a required to be excited when using the irradiation port 102a, and outputs the information specifying the selected quadrupole electromagnets to the electromagnet power supply control part 409. The electromagnet power supply control part 409 controls the electromagnet power supply 410 based on the input information for specifying the deflecting electromagnet and the quadrupole electromagnet and the energy of the beam in order to supply the electric power to the specified deflecting electromagnet and the specified quadrupole electromagnet, and thus makes the deflecting electromagnet and the quadrupole electromagnet excited. The electric power level required at the deflecting electromagnet and the quadrupole electromagnet is linked to the energy of the beam for the individual irradiation port and stored precedently in the electromagnet power supply control part 409, and the electromagnet power supply control part 409 controls the individual electromagnet power supply 410 based on the electric power level stored in the electromagnet power supply control part 409. In this case, as for the electromagnets not required to be excited, their electric power level may be defined to be 0 or their electromagnet power supply 410 may be controlled so as to be shutdown.

As for the patient data captured by the patient data capture part 403, the current value to be applied to the wobbler electromagnet is put into the wobbler electromagnet power supply control part 411, and the inclination of the therapy bed is put into the therapy bed control part 412. The energy of the beam ejected from the synchrotron in the patient data captured by the patient data capture part 403 is put into the beam energy setup part 413. The wobbler electromagnet power supply control part 411 controls the wobbler electromagnet power supply 414 so as to supply the specified current value. Alternately, the therapy bed control part 412 controls the therapy bed drive equipment 415 so that the therapy bed may be inclined at the specified angle. The beam energy setup part 413 puts out the specified energy of the beam to the accelerator control equipment 416, and the accelerator control equipment 416 controls the synchrotron 20 so that the ejected beam may have the specified energy of the beam. It is allowed that the inclination of the therapy bed may be adjusted by the instruction provided by the operator through the input equipment 401.

As in the above-mentioned manner, the setup operation for irradiating the beam from the irradiation port 102 of the irradiation equipment 10a is completed. Upon the operator's input of the instruction for initiating the therapy through the input equipment 401 after completing the setup operations, the decision part 402 receiving this instruction puts out the ejection initiation instruction to the accelerator control part 416. The accelerator control equipment 416 receiving the ejection initiation instruction makes the synchrotron 20 ejects the beam, and then the beam ejected from the synchrotron 20 is led to the irradiation equipment 10a and irradiated from the irradiation port 102a to the patient. The radiation dose of the beam to be irradiated onto the diseased part is measured by the radiation dosimeter installed at the irradiation nozzle part, in which once its measured dose value reaches a predefined value, the ejection of the beam from the synchrotron 20 is made stop and thus the irradiation of the beam onto the diseased part is made stop.

As in the above-described manner, after completing the irradiation of the beam in the irradiation direction for the first irradiation operation, the irradiation of the beam is performed in the irradiation direction for the second irradiation operation. Though the second irradiation operation is similar to the case for the first irradiation direction and its detail description is not repeated here, if the irradiation port is altered when changing the irradiation direction, the position of the individual irradiation nozzle, the inclination of the therapy bed, the excited deflecting electromagnet and the excited quadrupole electromagnet are modified as well as the scatterer disposed at the individual irradiation nozzle part, the range modulator, the patient collimator and the patient bolus are replaced. Thus, the irradiation of the beam is repeated until all the irradiation in the setup directions are completed, and then, the irradiation of the beam in plural directions to the diseased part of the patient is conducted.

According to the above-mentioned embodiment of the present invention, as the irradiation nozzle parts 105a to 105c are shared by the plural irradiation ports 102a to 102c, it will be appreciated that the number of irradiation nozzles can be reduced and the cost of the irradiation equipment can be reduced in comparison with the case in which impendent nozzles are installed individually for the plural irradiation nozzles. In this embodiment, as the irradiation nozzle is composed of three irradiation nozzle parts 105a to 105c and separate drive equipment are installed individually for the irradiation nozzles and made move independently, it will be appreciated that the load for the individual drive equipment can be reduced in comparison with the case in which all the irradiation nozzles are driven by a single drive equipment. IN addition, as the equipment such as the patient collimator 505 and the patient bolus 506, which are required to be replaced every time when the irradiation direction is altered, can be moved independently and separately from the other equipment, it will be appreciated that the space for accessing to the equipment for its field replacement can be easily reserved, and the replacement work for the equipment may be performed smoothly.

Preferably, in the above-described embodiment, as for the equipment such as the patient collimator 505 and the patient bolus 506 that requires higher positioning accuracy, some moving mechanism with higher mechanical accuracy in comparison with the other equipment may be installed. Preferably, as for the heavier equipment such as the wobbler electromagnets 501a and 501b, the rigidity of their drive equipment may be made higher than the other equipment. In addition, in case of the therapy with the therapy bed 108 being inclined, when applying the medical treatment to the neighboring area of the internal organs the position of which is likely to move due to the inclination of the therapy bed 108, it is preferable to capture the image of the diseased part by the X-ray CT scanner after fixing the inclination of the therapy bed 108, and to define the patient data based on the image data obtained for this arrangement.

In the above-described embodiment, though Wobbler method in which though a couple of wobbler electromagnets and a couple of scatterers are used is illustrated, the present invention can be similarly applicable to the cases of using Double Scatterer method in which a couple of scatteres are used or Beam Scanning method in which the beam is scanned within the diseased part. It is allowed to determine the number of quadrupole electromagnets installed at the irradiation equipment so as to optimize the beam parameters for the diseased part, and thus the number of quadrupole electromagnets is not limited to the number illustrated in this embodiment. The equipment illustrated in this embodiment which are installed at the irradiation nozzle are shown as reference, and it is allowed that a horizontality monitor for measuring the horizontality of the beam and a range adjuster for adjusting the reachable depth of the beam by controlling the energy of the beam may be installed at the irradiation nozzle. Some equipment, if any, other than wobbler electromagnets which can be installed at the irradiation nozzle and can be controlled automatically may be controlled by the irradiation control equipment 400. Still in this embodiment, though a couple of irradiation equipment is illustrated so as to be arranged for a single synchrotron 20, the number of irradiation equipment is not limited to 2. In addition to the irradiation equipment illustrated in this embodiment, it is allowed to install a rotational gantry which may change the irradiation direction by rotating around the patient. Though this embodiment assumes such a case that three irradiation ports are arranged, the number of the irradiation port is not limited to 3 but it may be plural numbers such as 2, 4 or more. This means that even if the number of irradiation ports may change, the cost for the overall equipment can be reduced by sharing a single irradiation nozzle for plural irradiation ports.

(Embodiment 2)

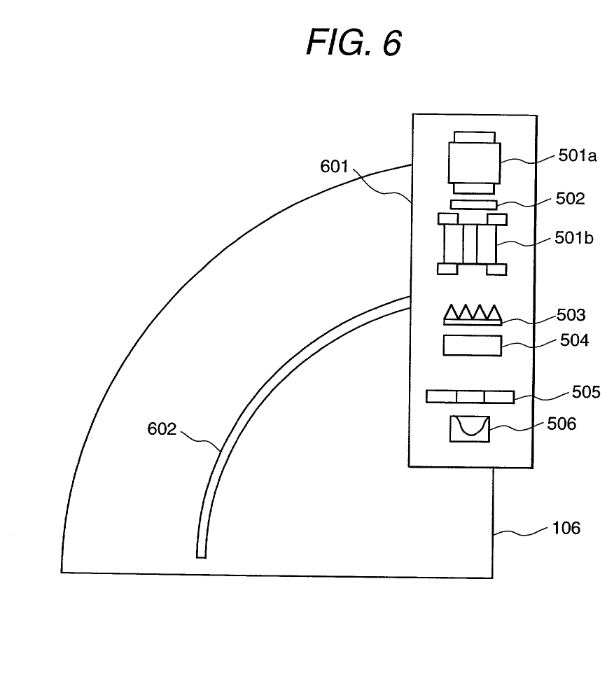

FIG. 6 illustrates an irradiation nozzle of the charged particle beam irradiation equipment as another embodiment of the present invention. As shown in the figure, in this embodiment, a single irradiation nozzle part 601 is formed as an integrated unit of the wobbler electromagnets 501a and 501b, the scatterer 502, the range modulator 503, the radiation dose monitor 504, the patient collimator 505 and the patient bolus 506, and the position for the individual equipment may be changed concurrently all together. In this configuration, a single rail 602 is installed at the support plate 106 and the irradiation nozzle 601 is moved by a single drive equipment. As this embodiment has the same structure as the embodiment 1 excluding that the number of drive equipment is 1, its detail description is not repeated here.

According to the embodiment 2, it will be appreciated that the number of drive equipment can be reduced and the cost of the overall drive equipment can be reduced in comparison with the embodiment 1.

What is claimed is:

1. A charged particle beam irradiation equipment comprising
plural irradiation ports providing a charged particle beam in plural different directions to an irradiation target to which a charged particle beam is irradiated, and a device for changing a characteristic of a charged particle beam provided from said irradiation port based on said irradiation target,
wherein said device is shared by said plural irradiation ports.

2. A charged particle beam irradiation equipment comprising
a first irradiation port providing a charged particle beam to an irradiation target to which a charged particle beam is irradiated, a second irradiation port providing a charged particle in a direction different from said first irradiation port to said irradiation target and a device for changing a characteristic of a charged particle beam provided from said irradiation port based on said irradiation target,
further comprising a moving mechanism for moving said device arranged between a position through which a charged particle beam provided from said first irradiation port passes and a position through which a charged particle beam provided from said second irradiation port.

3. A charged particle beam irradiation equipment of claim 1 or 2, wherein said device is either of a wobbler electromagnet, a scatterer, a range modulator, a patient collimator or a patient bolus.

4. A charged particle beam irradiation equipment comprising
a first irradiation port providing a charged particle beam to an irradiation target to which a charged particle beam is irradiated, a second irradiation port providing a charged particle in a direction different from said first irradiation port to said irradiation target and plural devices for changing a characteristic of a charged particle beam provided from said irradiation port based on said irradiation target,
wherein
said plural devices are divided into plural groups; and
said moving mechanism for moving said device between a position through which a charged particle beam provided from said first irradiation port passes and a position through which a charged particle beam provided from said second irradiation port is arranged individually for each of said plural groups.

5. A charged particle beam irradiation equipment of claim 4, wherein said plural devices include at least one of a wobbler electromagnet, a scatterer, a range modulator, a patient collimator and a patient bolus.

6. A charged particle beam irradiation equipment of claim 5, wherein said moving mechanism installed at a group including said patient collimator or said patient bolus has a positioning accuracy higher than a positioning accuracy of a moving mechanism installed at another group.

7. A charged particle beam irradiation equipment of claim 5, wherein said moving mechanism installed at a group including said wobbler electromagnet has a rigidity higher than a rigidity of the moving mechanism installed at another group.
